(19) United States
(12) Patent Application Publication
(10) Pub. No.: US 2015/0224249 A1
(43) Pub. Date: Aug. 13, 2015

(54) TISSUE ENUCLEATION DEVICES AND RELATED METHODS

(71) Applicant: Boston Scientific Scimed, Inc., Maple Grove, MN (US)

(72) Inventors: Ronald CIULLA, Westford, MA (US); Kenneth P. REEVER, Hopedale, MA (US); Kenneth J. DAIGNAULT, Holden, MA (US); Timothy Paul HARRAH, Cambridge, MA (US)

(73) Assignee: Boston Scientific Scimed, Inc.

(21) Appl. No.: 14/618,787

(22) Filed: Feb. 10, 2015

Related U.S. Application Data
(60) Provisional application No. 61/938,733, filed on Feb. 12, 2014.

(51) Int. Cl. A61M 5/00; A61K 51/00; A61B 18/20; A61N 5/06; A61B 17/32

(52) U.S. Cl. CPC A61M 5/007; A61N 5/0613; A61B 17/32002; A61B 18/20; A61K 51/00; A61N 2005/067

(57) ABSTRACT

A method for treating BPH may include marking a target tissue portion of a prostate of a patient with a contrasting agent. The method may further include emitting energy from an energy emitting device toward the target tissue portion. Further, the method may include enucleating the target tissue portion.

### CROSS REFERENCE TO RELATED APPLICATIONS

This application claims the benefit of U.S. Provisional Application No. 61/938,733, filed Feb. 12, 2014, the disclosure of which is incorporated herein by reference in its entirety.

### DESCRIPTION OF THE DISCLOSURE

1. Field of the Disclosure

Embodiments of the present disclosure relate generally to medical devices and procedures. In particular, embodiments of the present disclosure relate to medical devices and methods for tissue enucleation and extraction.

2. Background of the Disclosure

Benign Prostatic Hyperplasia (BPH) is noncancerous enlargement of the prostate gland in men. BPH includes hyperplasia (an increase in the number of cells) of prostatic stromal and epithelial cells which result in the formation of large nodules in the periurethral region of the prostate. As the prostate enlarges it puts pressure on and/or partially or completely occludes the urethra. Additionally, prostate enlargement may cause pain, difficulty in urination, infection, or the like.

Enucleation of the Prostate (EP) is a technique for treating BPH. EP typically involves inserting an energy emitting device into the urethra and directing the device to target tissue including enlarged prostate tissue. Typically, such energy emitting devices are directed to target tissue using a sheath such as, for example, a laserscope, a cystoscope, and/or rectoscope. The energy emitting device enucleates (e.g., separates or removes) the target prostate tissue away from its surroundings. Typically the separated prostate tissue may form one or more large pieces of tissue, referred to as “tissue balls”, which are then directed (e.g., pushed) into the bladder using the energy emitting device or another medical device. While referred to herein as a “ball,” the severed tissue may not necessarily be in the shape of a ball (e.g., sphere) but rather, may have any shape including irregular shapes. The energy emitting device is then removed and another device such as a morcellator or other extraction tool is introduced into the sheath for removing the tissue. A morcellator is a surgical device having a small opening at its distal end, one or more cutting blades, and suction capability. The blades may cut (e.g., mince, puree) the large pieces of tissue, e.g., tissue balls, that were moved into the bladder into smaller pieces. These smaller pieces may then be removed from the body through the opening via, suction and/or other means.

EP procedures often require great skill and typically are performed by very experienced medical professionals. The procedure is “blind,” in that incisions are made within the prostate without a readily ascertainable sense of the “depth” or geometry of the prostate. As such, extreme care must be used so as to not inadvertently harm (e.g., cut, damage, etc.) unintended portions of the prostate gland, including, for example, the prostatic capsule which lines the prostate gland. Such challenges result in a long learning curve for medical professionals before they become efficient with the EP technique. Accordingly, it may be desirable to provide for improved systems and methods for tissue enucleation and extraction.

### SUMMARY OF THE DISCLOSURE

Embodiments of the present disclosure include medical devices, such as an injection needle, that may be used to mark or indicate selective target tissue portions to be separated from remaining tissue portions, and methods of use thereof.

In one example, a method for treating BPH may be disclosed. The method may include marking a target tissue portion of a prostate of a patient with a contrasting agent. The method may further include emitting energy from an energy emitting device toward the target tissue portion. Further, the method may include enucleating the target tissue portion.

The medical device may further include one or more of the following features: the target tissue portion may include at least one of a lateral lobe and a medial lobe of the prostate; the contrasting agent may include at least one of iodine, barium, gadolinium, carbon particles in a fluid suspension, and methylene blue; the contrasting agent may include a radioactive tracer; injecting the target tissue portion with a needle; the target tissue portion may a first target tissue portion and the method may further include repositioning the needle and marking a second target tissue portion of the prostate with the contrasting agent; the energy emitting device may include a holmium:YAG laser; urging enucleated tissue into a bladder of the patient and extracting the enucleated tissue from the bladder of the patient via an extraction device; and the extraction device may include at least one of a morcellator and a high-pulse energy emitter.

In an additional or alternative example, a system for assisting in treating BPH is disclosed. The system may include an injection device. The injection device may include a needle tip which may be configured to penetrate a target tissue portion of a prostate. The injection device may further include a channel in fluid communication with one or more openings on a distal end of the injection device. The system may also include a source comprising a contrasting agent. The source may be fluidly coupled with the injection device such that, upon actuation, the contrasting agent may be delivered into the target tissue portion via the one or more openings. Further, the system may include an energy emitting device configured to emit energy toward the target tissue portion so as to enucleate the target tissue portion.

The system may further include one or more following features: the energy emitting device may include a holmium:YAG laser; the injection device may be configured to deliver the contrasting agent into the target tissue portion under pressure; and the target tissue portion may include at least one of a lateral lobe and a medial lobe of the prostate; the contrasting agent may include at least one of iodine, barium, gadolinium, carbon particles in a fluid suspension; and the contrasting agent may include a radioactive tracer.

In another additional or alternative example, a method for treating BPH may be disclosed. The method may include marking a target tissue portion of a prostate of a patient with a contrasting agent via an injection device. The injection device may be configured to penetrate the target tissue portion. Also, the target tissue portion may include at least one of a lateral lobe and a medial lobe of the prostate. The method may further include emitting laser energy from a holmium:YAG laser toward the target tissue portion so as to enucleate the target tissue portion.

The medical method may further include one or more of the following features: the contrasting agent may include at least one of iodine, barium, and gadolinium; the contrasting agent may include a radioactive tracer; the target tissue portion may be a first target tissue portion and the method may further include repositioning the injection device and marking a second target tissue portion of the prostate with the contrasting agent; and the method may further include urging enucleated tissue into a bladder of the patient and extracting the enucleated tissue from the bladder of the patient via an extraction device.

Additional objects and advantages of the present disclosure will be set forth in part in the description which follows, and in part will be understood from the description, or may be learned by practice of the claimed disclosure. The objects and advantages of the claimed disclosure will be realized and attained by means of the elements and combinations particularly pointed out in the appended claims.

It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 illustrates a male pelvic region with a healthy urinary system;

FIG. 2 illustrates a male pelvic region suffering from Benign Prostatic Hyperplasia (BPH);

FIG. 3 illustrates a cross-sectional top-view of a prostate suffering from BPH;

FIG. 4 illustrates an exemplary injection device according to a first embodiment;

FIG. 5 depicts an exemplary energy emitting device in vivo; and

FIG. 6 is a flow diagram illustrating an exemplary method of performing an enucleation procedure, according to a further embodiment of the present disclosure.

### DESCRIPTION OF THE EMBODIMENTS

Reference will now be made in detail to embodiments of the present disclosure, examples of which are illustrated in the accompanying drawings. Wherever possible, the same reference numbers will be used throughout the drawings to refer to the same or like parts. As used herein, the term “distal” refers to the direction that is away from the user and into the patient's body. By contrast, the term “proximal” refers to the direction that is closer to the user and away from the patient's body.

### Overview

Embodiments of the present disclosure may include a medical device for removing and/or retrieving a material from a body and methods of use thereof. In some embodiments, the medical device may be used to retrieve tissue that has been cut away or otherwise severed from its surroundings. In at least one embodiment, the tissue to be removed may be tissue from the prostate for treatment of BPH. In alternative embodiments, the medical device may be used to remove other types of tissues or materials such as, for example, bladder stones, kidney stones, and the like. For convenience, the exemplary medical devices discussed herein are referred to as an injection device, an energy emitting device, and an extraction device; however, these references are merely made for convenience, and are intended to include devices capable of other and/or additional operations and/or functions such as resection.

In the following sections, embodiments of the present disclosure will be described using the prostate as an exemplary body organ. It will be understood that the prostate is merely an example and that the disclosed devices may be utilized in other parts of the body.

The present disclosure provides medical devices for marking, enucleating, and removing tissue from a patient's body. The medical devices may be used to remove tissue that has been cut away or excised from the body. The disclosed devices may be configured to be introduced into the body through a suitable natural opening, such as through the urethra.

### EXEMPLARY EMBODIMENTS

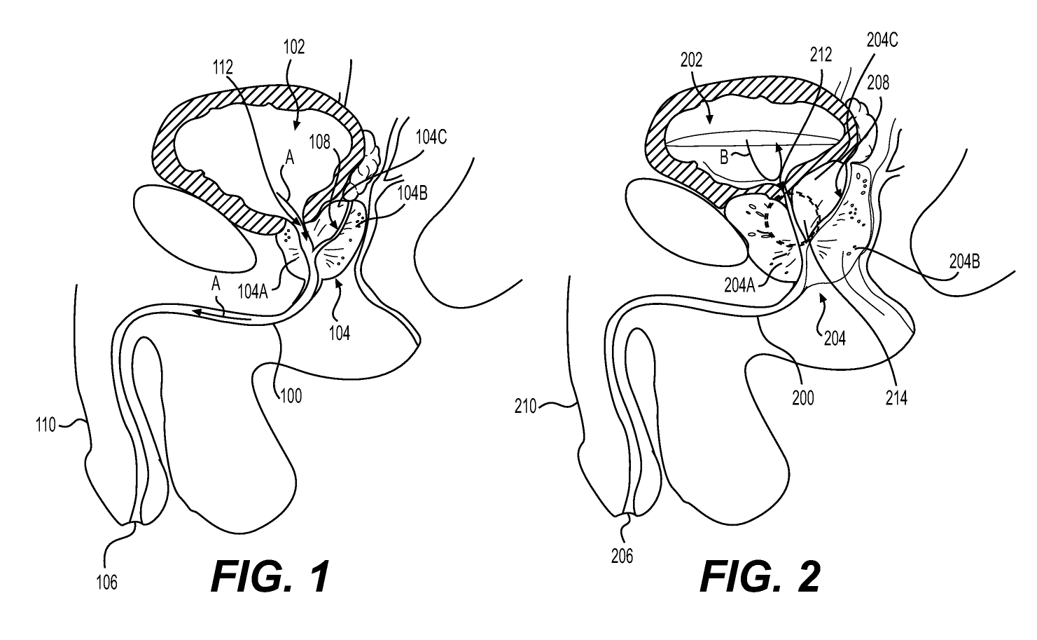

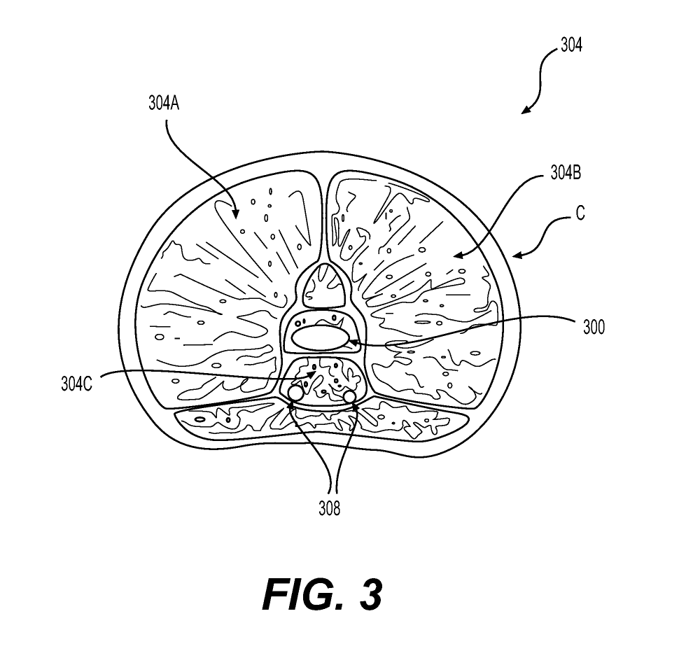

FIG. 1 illustrates the pelvic region of a male having a healthy urinary system. As shown in FIG. 1, the urinary system includes a urethra 100, a bladder 102, a prostate 104, a urinary meatus 106, and an ejaculatory duct 108 of the male urinary system. The urethra 100 is a biological lumen connecting bladder 102 to the urinary meatus 106 at the tip of the penis 110. The urethra 100 connects to the bladder 102 at bladder opening 112. As shown, the prostate 104 is positioned around the urethra 100 between the bladder 102 and the penis 110. The prostate 104 is comprised of multiple lobes, including two lateral lobes 104A and 104B, and a medial lobe 104C within a prostatic capsule “C” (FIG. 3). Upon stimulation, the bladder 102 constricts and urine (depicted by arrow “A”) is released out of the urinary meatus 106 through the urethra 100.

FIG. 2 illustrates the male pelvic region suffering from Benign Prostate Hyperplasia (BPH). As shown, the pelvic region includes a urethra 200, a bladder 202, a prostate 204, a urinary meatus 206, an ejaculatory duct 208, a penis 210, and a bladder opening 212. In BPH, the passage of urine through the urethra 200 from the bladder 202 to the urinary meatus 206 at the tip of the penis 210 is obstructed by the enlarged prostate 204. For example, an excess tissue region 214 in the enlarged prostate 204, for example, excess tissue within one or more of the lateral or medial lobes 204A, 204B, and 204C, may constrict (e.g., narrow, obstruct, and/or partially occlude) the urethra 200 proximate to the bladder opening 212, and may cause pain, difficulty in urination, and/or urinary infections. For example, due to constriction of the urethra, urine (depicted by arrow “B”) is prevented from freely flowing through the urethra 200 to exit the penis 210 via the urinary meatus 206. In turn, it may result in urine buildup in the bladder 202. This accumulation of urine may increase infection and the occurrence of other urinary tract problems. In addition, the enlarged prostate 204 may constrict (e.g., narrow, obstruct, and/or partially occlude) the ejaculatory duct 208 causing erectile dysfunction or ejaculatory problems.

The enlarged prostate 204 may be severed using various methods, such as, enucleation as is known in the art. In enucleation, an energy emitting device, e.g., a laser, is used to cut (e.g., sever, ablate) tissue from remaining portions of the enlarged prostate 204. By way of example only, enucleation may be used to cut excess tissue region 214 from the enlarged prostate 204 in order to alleviate constriction of the urethra 200. One such energy-based surgical procedure includes Enucleation of the Prostate (EP). In this procedure, an energy source such as, for example, a holmium:YAG (Ho:YAG) laser, is used to remove obstructive prostate tissue. The Ho:YAG surgical laser is a solid-state, pulsed laser that emits light at a wavelength of approximately 2100 nm. This wavelength of light is particularly useful for tissue ablation as it is strongly absorbed by water. An advantage of Ho:YAG lasers is that they can be used for both tissue cutting and for coagulation. Energy-based surgical procedures, such as EP, are becoming increasingly preferred over previous methods (e.g., drug therapy) due to fewer complications requiring shorter hospital stays, shorter catheterization times, and shorter recovery times. While certain embodiments of this disclosure refer to the use of a Ho:YAG surgical laser, it is understood that additional or alternative energy sources may be used. Such sources may include RF energy emitting devices and non-HO:YAG surgical lasers having any appropriate wavelength.

As noted previously, EP procedures may require great skill and typically are performed by very experienced medical professionals. The procedure is “blind” in that incisions are made within the prostate 304 without a readily ascertainable sense of the “depth” or geometry of the prostate 304. As such, extreme care must be used so as to not inadvertently harm (e.g., cut, damage, etc) unintended portions of the prostate 304, including, for example, the prostatic capsule C which lines the prostate 304, as shown in FIG. 3. During an EP procedure, a medical professional may insert an energy emitting device, e.g., a laser, into the urethra 300 of a patient and direct energy toward one or more of the lateral lobes 304A and/or 304B and/or the medial lobe 304C so as to cut (e.g., separate) tissue. Following each cut of tissue via the energy emitting device, additional layers of the prostate 304 may become exposed and available for enucleation. However, as the medical professional continues to advance the energy emitting device so as to enuculeate deeper tissue portions within the prostate, he or she runs the risk of exposing and/or cutting the prostatic capsule C which may cause serious damage to nerves running therethrough, and consequently, increased pain and/or hemorrhage for a patient. Accordingly, it may be desirable to provide improved methods and systems for defining cutting planes within a prostate 304 to guide a medical professional during a procedure.

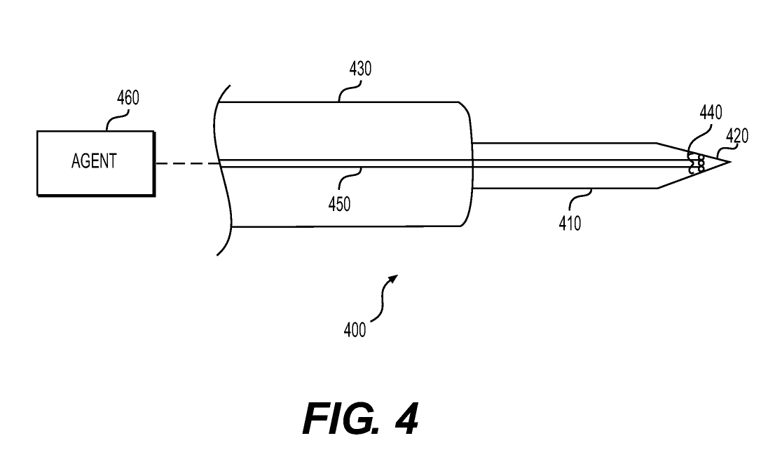

In an exemplary embodiment, as shown in FIG. 4, an injection device 400 may include a needle 410. Needle 410 may be any commercially available injection needle having a tip 420 configured to penetrate tissue. For example, tip 420 may be sharpened to facilitate insertion of needle 410 into a target tissue portion. Needle 410 may also include an elongate member 430 configured for insertion into a lumen of a scope device (e.g., a cystescope, rectoscope, or the like). For example, elongate member 430 may have an outer diameter smaller than a diameter of a lumen (e.g., working channel) of an appropriate scope device. Accordingly, needle 410 may be readily inserted into a body via a scope device. Additionally, the elongate member 430 and/or a lumen of the scope device may be coated with or comprised of a lubricious material to facilitate smooth insertion of the needle 410 through the lumen of the scope device.

Needle 410 may also include one or more openings 440 configured for passing an agent therethrough. The one or more openings 440 may be in fluid communication with a fluid passage 450 extending through elongate member 430 and needle 410. As shown in FIG. 4, fluid passage 450 may be configured to convey an agent from a source 460 through the one or more openings 440 upon actuation (e.g., via an actuator) by a user. In some embodiments, an agent may be delivered out of the one or more openings 440 under pressure. It is understood that various modifications of the injection device 400 may be made without departing from the scope of this disclosure. For example, tip 420 may be curved, bent, or otherwise modified to facilitate penetration of tip 420 into and/or through tissue. Additionally or alternatively, needle 410 and/or elongate member 430 may have any cross-sectional shape appropriate for passage through the lumen of the scope device. For example, circular, oval, irregular, etc., cross-sectional shapes may be used. Additionally, the sizes (e.g., length, etc.) and/or dimensions (e.g., outermost diameter, etc.) of the injection device 400 may be selected based on, at least in part, the particular patient anatomy and/or desired use. Further, the arrangement, position, and/or number of openings 440 may be altered without departing from the scope of the disclosure.

In at least some exemplary embodiments, a cystescope (not shown) may be introduced into the body of a patient. For example, the cystescope may be introduced into a urethra 300 (FIG. 3) of a patient. The cystescope may include a vision system configured to aid a medical professional with accurate placement of one or more tools (e.g., injection device 400) within the body of the patient. The vision system may include lenses to focus an image, one or more optical fibers for transmitting light and/or images from a distal end of the cystescope to a viewing piece at a proximal end of the cystescope or a display device operably coupled with the one or more optical fibers, a vision chip (e.g., CCD, CMOS, etc.), a camera, and/or an illumination device to illuminate an interior of the body of a patient. After insertion of the cystescope into the urethra 300 of a patient, injection device 400 may be inserted through the lumen of the cystescope such that a distal end of the injection device extends distally of the distal end of the cystescope. Upon proper placement, injection device 400 may be actuated such that the tip 420 penetrates through a wall of the urethra 300 and is directed into one of the lobes 304A-C of the prostate 304. After insertion of the tip 420 into the selected lobe 304A-C, a medical professional, with the aid of the vision system of the cystescope, may inject an agent into the selected lobe 304A-C from source 460 through fluid channel 450 and out of the one or more openings 440. The medical professional may then, if desired, reposition the injection device 400 so as to penetrate and inject another one of the lobes 304A-C of the prostrate 304.

The agent may include a solution configured to accurately discriminate between target prostate tissue in the lobes 304A-C and the capsule C of the prostate. For example, the agent may include a contrast material/dye. Such materials include substances used to make specific organs, blood vessels, or types of tissue (e.g., prostate lobes 304A-C) more visible on X-rays. Common contrast material substances include iodine, barium, and gadolinium. Additionally or alternatively, the agent may include a quantity of radioactive tracer or dye. Because of the tracer's biochemistry, the lobes 304A-C may collect more of the tracer than does the surrounding capsule C. Thus, when the radioactive tracer decays and emits gamma rays, a higher number of these gamma rays will originate from the lobes 304A-C than from equal volumes of the capsule C. The tracer distribution and gamma ray emission can be identified using standard imaging techniques including, e.g., fiber optics, etc. In some exemplary embodiments, the agent may comprise a dye including optical contrast pigments such as, for example, carbon particles and/or methylene blue. Additionally or alternatively, in some exemplary embodiments, the agent may include a solution configured to assist in the cessation of bleeding of tissue. Regardless of the specific agent used, upon injection of the agent into selected portions of the lobes 304A-C, a medical professional may more readily ascertain cutting planes within the prostate 304.

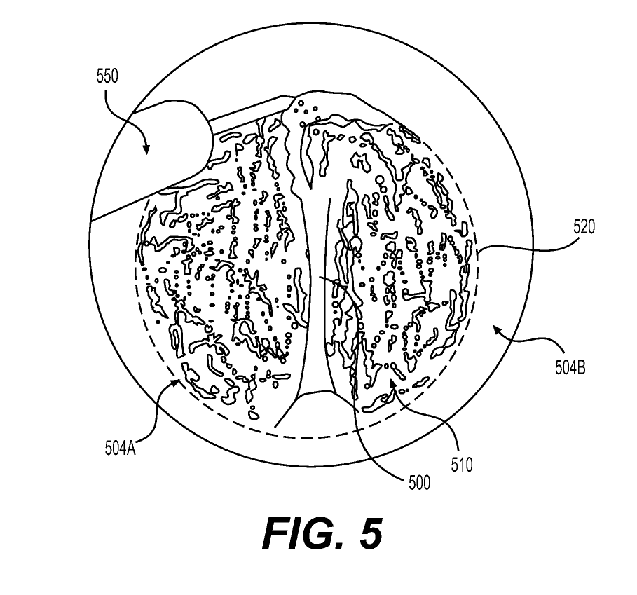

As shown in FIG. 5, an exemplary portion of a prostate 504 has been injected with an agent 510. For example, as shown in FIG. 5, a target tissue portion 520 including tissue within both of the lateral lobes 504A and 504B has been injected with an agent 510 such that portions of the lobes 504A and 504B are now tinted, dyed, stained, or otherwise distinguishable from remaining portions of the prostate 504. As shown in FIG. 5, each of the lateral lobes 504A and 504B have been injected with a suitable agent 510. However, other embodiments may include injection of the medial lobe 504C (not shown), injection into only a single lobe 504A-C, or injection into one of lateral lobes 504A and 504B and injection into medial lobe 504C.

Once a target tissue portion 520 has been injected with agent 510, injection device 400 and scope device (e.g., cystescope or rectoscope) may be removed from the patient. Next, a laserscope (not shown) may be inserted into the urethra of the patient. Any readily available and appropriately sized laserscope may be used. The laserscope may include a lumen (e.g., working channel) through which an energy emitting device 550 (e.g., a holmium:YAG (Ho:YAG) laser) may be passed. For example, in some embodiments, the energy emitting device 550 and/or a lumen of the laserscope may be coated with or comprised of a lubricious material to facilitate smooth insertion of the energy emitting device 550 through the lumen of the laserscope. Additionally, the laserscope may include a vision system configured to aid a medical professional with accurate placement of the energy emitting device 550 within the body of the patient. The vision system may include lenses to focus an image, one or more optical fibers for transmitting light and/or images from a distal end of the laserscope to a viewing piece at a proximal end of the laserscope or a display device operably coupled with the one or more optical fibers, a vision chip (e.g., CCD, CMOS, etc.), a camera, and/or an illumination device to illuminate an interior of the body of a patient.

Once the laserscope is positioned within the urethra of the patient, the energy emitting device 550 may be inserted through the lumen of the laserscope such that a distal end of the energy emitting device is adjacent the target tissue portion 520. As noted above, the laserscope may include a vision system to guide a medical professional during placement of the laserscope adjacent the target tissue portion 520. After positioning, the medical professional may activate the energy emitting device 550 and direct laser energy emitted therefrom toward the target tissue portion 520. By virtue of agent 510, the target tissue portion 520 may be readily distinguishable from remaining portions of the prostate, including the capsule C on the laserscope vision system. Accordingly, a medical professional may quickly and confidently direct the energy emitted from energy emitting device 550 without harming the capsule C. Accordingly, the disclosed devices and methods enable increasingly efficient and safer enucleation of a patient's prostate.

Enucleating, cutting, and/or severing of target tissue portion 520 may form a tissue ball. Once formed, the tissue ball may be moved (e.g., pushed) into the bladder 202 (FIG. 2) for removal via an appropriate tool. For example, in some embodiments, a rectoscope or other scope device may be inserted into the patient's urethra for introduction of a tissue extraction device. Such extraction devices may include, for example, a high-pulse energy emitter to destroy the tissue ball, or a morcellating tool to cut the tissue ball into small pieces.

While embodiments described herein include the introduction of multiple scope devices, e.g., a rectoscope, cystescope, and/or a laserscope, it is understood that in some embodiments, a single fully equipped scope device may be used to introduce injection device 400, energy emitting device 550, and a tissue extractor (not shown) without departing from the scope of the disclosure.

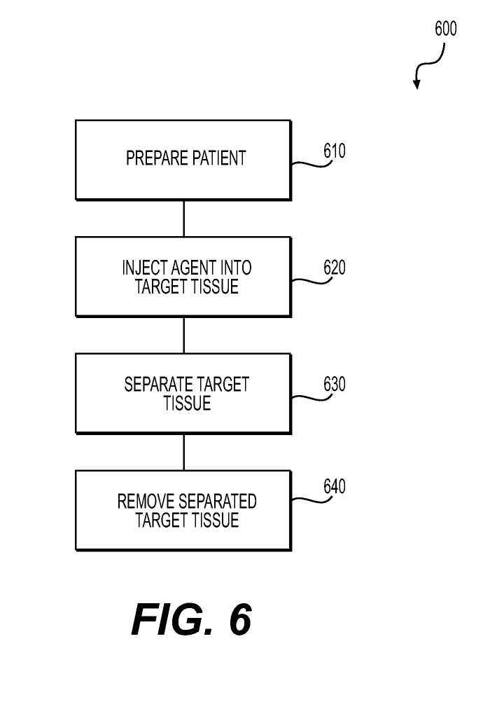

FIG. 6 illustrates an exemplary method 600 for performing an enucleation procedure. First, a patient may be prepared for the procedure at step 610. Such preparation may include administering anesthetics or other pain blocking medicines and/or devices, positioning, sterilizing, and/or draping the patient as is known in the art. Following preparation of the patient, target tissue may be injected with an agent at step 620. As discussed above, a scope device (e.g., rectoscope, cystescope, etc.) may be used to facilitate insertion of an injection device 400 into a urethra 300 of a patient. Once positioned, a tip 420 of the injection device 400 may be actuated so as to penetrate a wall of the urethra 300 and may be directed into one of the lobes 304A-C of the prostate 304. After insertion of the tip 420 into the selected lobe 304A-C, a medical professional, with the aid of the vision system of the cystescope, may inject an agent into the selected lobe 304A-C from source 460 through fluid channel 450 and out of the one or more openings 440. The medical professional may then, if desired, reposition the injection device 400 so as to penetrate another one of the lobes 304A-C of the prostrate 304. At each injection site, a few cubic centimeters (cc's) may be injected as appropriate.

At step 630, a laserscope may be positioned within the urethra of the patient, and the energy emitting device 550 may be inserted through the lumen of the laserscope such that a distal end of the energy emitting device is adjacent the target tissue portion 520. After positioning, the medical professional may activate the energy emitting device 550 and direct energy, e.g., laser energy, emitted therefrom toward the target tissue portion 520 so as to enucleate (e.g., separate, cut, etc.) the target tissue portion 520 from the remaining portion of the prostate. In so doing, the medical professional may direct the energy emitting device 550 towards those portions of the prostate identified with agent 510. Indeed, the injected agent 510 may guide the medical professional to the target tissue portion 520 to be enucleated, while remaining portions of the prostate are left unharmed. The enucleated tissue may then be pushed into the bladder for subsequent removal at step 640. For example, the enucleating tissue may be removed via any appropriate tissue extraction device including, for example, a high-pulse energy emitter and/or a morcellating device.

Using the exemplary methods described herein, a medical professional may readily and accurately remove enlarged prostatic tissue from a patient's body for treatment or diagnostic purposes. It is understood, however, that while embodiments of the present disclosure have been described in reference to treating BPH, the teachings of the present disclosure may be used to treat other tissues and/or disorders. Further, the devices herein may be single-use which can be discarded after one use or may be used again after sterilization.

Other embodiments of the present disclosure will be apparent to those skilled in the art after consideration of the specification and practice of the embodiments disclosed herein. It is intended that the specification and examples be considered as exemplary only, with a true scope and spirit of the disclosure being indicated by the following claims.

What is claimed is:

1. A method for treating BPH, comprising:
marking a target tissue portion of a prostate of a patient with a contrasting agent; and
emitting energy from an energy emitting device toward the target tissue portion so as to enucleate the target tissue portion.

2. The method of claim 1, wherein the target tissue portion includes at least one of a lateral lobe and a medial lobe of the prostate.

3. The method of claim 1, wherein the contrasting agent includes at least one of iodine, barium, gadolinium, carbon particles in a fluid suspension, and methylene blue.

4. The method of claim 1, wherein the contrasting agent includes a radioactive tracer.

5. The method of claim 1, further including:
injecting the target tissue portion with a needle.

6. The method of claim 5, wherein the target tissue portion is a first target tissue portion, the method further including:
repositioning the needle; and
marking a second target tissue portion of the prostate with the contrasting agent.

7. The method of claim 1, wherein the energy emitting device includes a holmium:YAG laser.

8. The method of claim 1, further including:
urging enucleated tissue into a bladder of the patient; and
extracting the enucleated tissue from the bladder of the patient via an extraction device.

9. The method of claim 8, wherein the extraction device includes at least one of a morcellator and a high-pulse energy emitter.

10. A system for assisting in treating BPH, comprising:
an injection device, including:
a needle tip configured to penetrate a target tissue portion of a prostate; and
a channel in fluid communication with one or more openings on a distal end of the injection device;
a source comprising a contrasting agent, the source being fluidly coupled with the injection device such that, upon actuation, the contrasting agent is delivered into the target tissue portion via the one or more openings; and
an energy emitting device configured to emit energy toward the target tissue portion so as to enucleate the target tissue portion.

11. The system of claim 10, wherein the energy emitting device includes a holmium:YAG laser.

12. The system of claim 10, wherein the injection device is configured to deliver the contrasting agent into the target tissue portion under pressure.

13. The system of claim 10, wherein the target tissue portion includes at least one of a lateral lobe and a medial lobe of the prostate.

14. The system of claim 10, wherein the contrasting agent includes at least one of iodine, barium, gadolinium, carbon particles in a fluid suspension, and methylene blue.

15. The system of claim 10, wherein the contrasting agent includes a radioactive tracer.

16. A method for treating BPH, comprising:
marking a target tissue portion of a prostate of a patient with a contrasting agent via an injection device configured to penetrate the target tissue portion, wherein the target tissue portion includes at least one of a lateral lobe and a medial lobe of the prostate; and
emitting laser energy from a holmium:YAG laser toward the target tissue portion so as to enucleate the target tissue portion.

17. The method of claim 16, wherein the contrasting agent includes at least one of iodine, barium, and gadolinium.

18. The method of claim 16, wherein the contrasting agent includes a radioactive tracer.

19. The method of claim 16, wherein the target tissue portion is a first target tissue portion, the method further including:
repositioning the injection device; and
marking a second target tissue portion of the prostate with the contrasting agent.

20. The method of claim 16, further including:
urging enucleated tissue into a bladder of the patient; and
extracting the enucleated tissue from the bladder of the patient via an extraction device.
